(19) United States
(12) Patent Application Publication
(10) Pub. No.: US 2020/0386739 A1
(43) Pub. Date: Dec. 10, 2020

(54) TREATMENT OF METAHNOGENIC BACTERIA AS A THERAPY FOR CHRONIC LUNG DISEASE

(71) Applicant: Edy Edmon Soffer, Los Angeles, CA (US)

(72) Inventor: Edy Edmon Soffer, Los Angeles, CA (US)

(21) Appl. No.: 16/892,060

(22) Filed: Jun. 3, 2020

Related U.S. Application Data
(60) Provisional application No. 62/856,747, filed on Jun. 4, 2019.

(51) Int. Cl. G01N 33/497

(52) U.S. Cl. CPC G01N 33/497; G01N 2800/52; G01N 2033/4975

(57) ABSTRACT

A method improves a symptom or indicator of disease in a patient with chronic lung disease. The patient is identified as likely to respond to treatment by reduction of methane production and/or methanogenic archaea in the digestive tract by evaluating the patient as methane positive. One or more agents is administered to the identified methane positive patient. The agents reduce or eliminate the methane production and/or the methanogenic archaea.

### RELATED APPLICATION

The present application claims priority under 35 U.S.C. § 119(e) to U.S. Provisional Application No. 62/856,747, filed Jun. 4, 2019.

### FIELD OF INVENTION

The present invention relates generally to the treatment of pulmonary diseases, and more particularly to treatments based on reducing or eliminating methanogenic bacteria in the gut and reducing or eliminating the level of methane exhaled by the lungs

### BACKGROUND OF THE INVENTION

The following description includes information that may be useful in understanding the present invention. It is not an admission that any of the information provided herein is prior art or relevant to the presently claimed invention, or that any publication specifically or implicitly referenced is prior art.

Asthma, chronic obstructive lung disease (COPD) and asthma—COPD overlap are common respiratory conditions, characterized by airway limitation, and present typically with shortness of breath, cough and/or wheezing (1-4). Pulmonary function tests measure lung volumes capacity, rates of flow, and gas exchange and are among the most important diagnostic tools in the evaluation of respiratory conditions (5). Pharmacological therapy consists primarily of bronchodilators and anti inflammatory agents (1,3)

The gastrointestinal tract in humans, primarily the colon and the distal part of the small bowel, is populated by a large number of microorganisms, which include bacteria, archea, viruses, and unicellular eukaryotes (6), that live in symbiosis with the host and are involved in various homeostatic processes (6,7). Methanogens, methane producing microorganisms, generally belong to the domain archaea, and live in strictly anaerobic conditions (23) primarily in the large bowel. They are difficult to cultivate. Because their cell wall lacks peptidoglycan they are susceptible only to certain antibiotics (i.e., bacitracin and chloramphenicol) and resistant to many common others like penicillins and aminoglycosides (24). Archaea are the only confirmed biological sources of methane in nature and Methanobrevibacter smithii is the predominant methanogen in the human intestine (20), followed by Methanospaera stadmagnae (25), whereas Methannobrevibacter oralis which is implicated in periodontal disease is the main archaeon in the oral cavity (26).

The prevalence of methanogenic bacteria in humans varies, depending on the diagnostic method. Using either molecular (25) or culture techniques (27,10) methanogens account for approximately 10% of the human colon anaerobes. However, modern polymerase chain reaction (PCR) techniques detect the presence of 80% to almost 100% colonization of the human colon by M. smithii has been detected 28,11). Once produced, intestinal methane can be excreted either in the flatus or exhaled after traversing the gut mucosa and entering the systemic circulation without any further metabolism (29). Twenty (30) to 50% (31) of the methane produced is excreted in the exhaled breath, allowing for indirect measurement of CH₄ production in the intestine by breath testing.

Methane gas (CH₄) is produced using H₂ and CO₂ during the process of fermentation of the undigested polysaccharide fraction of certain carbohydrates (8). Methane then is mostly excreted in flatus, while some of it is also excreted through the lungs, during expiration (9). The presence of methanogens can be detected by analysis of colonic contents, using culture or molecular analysis techniques (10,11). Breath analysis determine the level of methane in expired air, and is the method of choice in clinical setting. The prevalence of methane production in the healthy population varies greatly, and depends in part on the cut-off concentration in the breath sample, ranging from >1 part per million (ppm) above the atmospheric concentration to >10 ppm any time during the test (12,13).

### SUMMARY

In one aspect, the present disclosure relates to a method of improving a symptom or indicator of disease in a patient with chronic lung disease. Examples of indicators of a disease that can be improved include reduction or elimination of methane gas in the patient's breath or results of a pulmonary function test. Examples of symptoms that can be improved include shortness of breath, cough and wheezing.

In this aspect, the method includes identifying the patient as likely to respond to treatment by reduction of methane production and/or methanogenic archaea in the patient's digestive tract by evaluating the patient as methane positive, and administering one or more agents to the identified methane positive patient, wherein the one or more agents reduce or eliminate the methane production and/or the methanogenic archaea in the patient's digestive tract. Thus, the method can reduce methane levels in the subject, which can be assessed by breath testing, or can reduce methanogenic archaea in the patient's intestine. The method can often reduce or eliminate the use of rescue medication by the subject.

One way to evaluate the patient as methane positive is by identifying 10 ppm or greater of methane in the patient's breath. It can be useful to evaluate intestinal methanogenic archaea or breath methane, before, after or during treatment.

Agents that can be administered include a probiotic, a prebiotic, an elemental, semi-elemental or polymeric formula. The agent can be present in a food. Another agent that can be administered is an antibiotic that is poorly absorbed systemically, such as rifaximin or an aminoglycoside. The agent can be administered daily, for period such as at least 5 days. Administering the agent can be repeated after stopping treatment for at least one day or longer.

### DETAILED DESCRIPTION

The following embodiments and aspects thereof are described and illustrated in conjunction with compositions and methods which are meant to be exemplary and illustrative, not limiting in scope.

The data relating to methane and the gut suggest that methane can modulate neuromuscular function in the gastrointestinal tract in health and disease. Translational studies suggest that methane acts like a neuromuscular transmitter resulting in increased contractile activity, but reduced propagation of the peristaltic movement in the intestine. Human studies showed that CH₄ production (measured by breath testing) delays transit time and also an association between methane status on breath testing with delayed transit associated conditions like constipation predominant IBS and chronic constipation. There is also preliminary evidence that antibiotic treatment results in improvement of symptoms in a certain proportion of patients suffering from these disorders in a fashion related to its ability to eradicate methane (eradication studies).

Recent data, derived from animal models and humans, suggest that methane is not just an inert gas, but a compound that can modulate motor activity and transit in the gut, in vivo infusion of methane into the small intestine of dogs, significantly slowed small intestinal transit time by 59%, as assessed by scintigraphy (14). Exposure of guinea pig ileum to methane significantly decreased peristaltic velocity, increased the amplitude of contractions, and increased the area under the curve (AUC) of intraluminal pressure (15). In a study of guinea pig muscle strips from the ileum, methane infusion increased contractile amplitude, blocked by tetrodotoxin and reduced by atropine, suggesting that the effects of methane on the intestine are mediated by the cholinergic pathway of the enteric nervous system (16). A number of studies in healthy subjects, constipation predominant irritable bowel syndrome (IBS-C) and patients with chronic constipation showed an association between the presence of methane, slow gut transit and symptoms (17,18). Furthermore, antibiotic therapy resulting in elimination of methane excretion is associated with improved symptoms in patients with IBS-C (18). In a recent study of methane producing patients with IBS-C and chronic constipation, treatment with the antibiotic rifaximin reduced methane production and resulted in an improvement of colonic transit time and constipation symptoms (19). Epidemiological data indicate that the prevalence of methane producers was higher in the slow transit compared to the normal transit (chronic constipated patients and also to healthy controls (38,39). The relationship between constipation and methane production was assessed in a detailed nested study that evaluated constipation severity using both subjective (patient's perception) and objective (number of bowel movements and Bristol stool score) measures in 87 Rome I IBS patients. In this study, constipation severity was significantly related to methane producer status and to the quantity of methane production during the lactulose breath testing, whereas methane production was correlated to a lower number of bowel movements and Bristol stool score (40). The level of methane detected in the breath, following a glucose test dose, in a large number of subjects with various functional GI symptoms correlated with severity of symptoms in patients with constipation (41). Data concerning therapy of methane producers are limited. A retrospective analysis of treatment data among C-IBS patients showed that the combination of the neomycin and rifaximin induced clinical improvement and in eliminating methane for methane producers (85% and 87%, respectively), and was more effective than either drug given alone (42).

Comparable to the structure of the gastrointestinal tract, the wall of the bronchioles, the tiny airways leading to the pulmonary alveoli, the tiny air sacs in the lung that allow for gaseous exchange, is also lined with smooth muscle that can contract and relax. Given the contracting effect of methane on smooth muscle in the gut and its excretion in the lungs, it was postulated that methane may induce smooth muscle contraction in the bronchioles. If so, production of methane in subjects who harbor methanogens (methane producing microorganisms) may induce/aggravate symptoms of patients with chronic lung disease; typical examples, though not limited to, being asthma and COPD. In a study of healthy subjects and patients with chronic lung disease (asthma, COPD, asthma-COPD overlap (partial asthma) and restrictive lung disease we found that methane production, as determined by breath test, was significantly more prevalent among patients with chronic lung disease as compared to healthy subjects as depicted in the enclosed table 1 (unpublished data).

The inventor has unexpectedly discovered that many forms of chronic lung disorders and diseases are associated with elevated levels of methane production in the digestive tract. To show that methane production and its expiration in the lung is associated with the pathogenesis of symptoms in patients with chronic lung disease, the prevalence of methane production in patients with chronic lung disease and in healthy subjects was evaluated. Patients with asthma, COPD and asthma—COPD overlap (partial asthma) and restrictive lung disease, as defined by metrics of their pulmonary function tests and according to established criteria, were recruited from a pulmonary clinic. Patients provided a breath sample, taken as a single spot collection. Gas was analyzed by QuinTron Breath Tracker device (QuinTron Instrument Company, Inc., Milwaukee, Wis.). A group of healthy volunteers underwent the same breath collection and analysis, and served as the control population.

Methane breath testing positivity is variably defined in the literature depending on the substrate and the cut-off used: it ranges from >1 part per million (ppm) above the atmospheric concentration (1.8 ppm) (29,12), to ≥3 ppm (21,22) and >10 ppm (23) any time during the test. Based on breath test results, individuals are categorized as methane producers and non-producers, accounting approximately for the 35% and 65% of a healthy Western population, respectively (29), and the proportion of methane producers remained stable over 35 years between 1970 and 2005 (12). A prevalence of 54% has been described by analyzing baseline CH₄ levels (34). A recent consensus recommended that methane levels ≥10 ppm. be considered as methane positive (35). An absence of methane in the breath does not mean absence of methanogenic flora. However, it seems that there is a threshold of approximately 10⁸ methanogens per gram dry weight of stool in order to detect the generated methane in breath sample (36); and methane producers harbor 10⁷ to 10¹⁰ methanogens per gram dry weight of stool (37). For the purpose of the study, methane levels below 10 ppm were used to denote a “methane negative” subject and levels above 10 ppm a “methane positive” one. The methane status of the subjects was compared between the two groups and the results are shown in Table 1.

TABLE 1

| | | Methane | | Methane | | |
|---|---|---|---|---|---|---|
| | | <10ppm | | ≥10ppm | | Fisher' s |
| | | N | % | N | % | exact P |
| Group 5 | Control | 88 | 89.8 | 10 | 10.2 | |
| | Asthma | 39 | 76.0 | 11 | 22.0 | .079 |
| | Partial asthma | 16 | 76.2 | 5 | 23.8 | .139 |
| | Restrictive | 34 | 79.1 | 9 | 20.9 | .109 |
| | COPD | 29 | 72.5 | 11 | 27.5 | .017 |
| | Any lung disease | 118 | 76.6 | 36 | 23.4 | .019 |

All p-values 2-sided are versus control. N denotes the number of subjects in each group

It can be seen that a statistically significant increase in methane positive patients was identified compared to control when results were pooled for any lung disease. Without wishing to be bound by any particular theory, it is believed that the methane production can either cause or aggravate the various lung disorders and diseases exhibited by patients. Thus, various embodiments of the present invention provide for identifying patients who are considered “methane positive” by breath test and can benefit from the method of the present invention. As seen above, in one embodiment, a breath methane level of at least 10 parts per million (ppm) is considered to be associated with aggravation of chronic lung symptoms. In further embodiments this level could be 1 ppm or any value higher than 1 ppm.

Various embodiments of the present invention provide for identifying patients likely to respond to treatment by reduction of methanogens and/or methanogeneis by identifying the presence of methane level in the breath, or presence of methanogens in biological samples. In addition to the assessment techniques described above, assessment of breath methane level can be conducted using other testing analysis methods and equipment known in the art. Additional examples of such equipment include, for example, the GASTROLYZER device produced by Bedfont Scientific of Harrietsam, United Kingdom and the QuinTron BreathTracker gas chromatographic (GC) analyzer. In addition to a single, spot collection, analysis could consist of a number of breath samples consisting of a baseline sample, collected after fasting, followed by, but not limited to, ingestion of a carbohydrate such as lactulose. Collections can be repeated every 5 minutes to every day, such as, but not limited to every 5 minutes, 15 minutes, 90 minutes, 120 minutes, 150 minutes, 240 minutes or 1440 minutes. In various embodiments, other carbohydrates, such as lactose, glucose or Xylose can replace lactulose. Peak methane level or area under the curve of the breath methane can be used to assess breath methane level.

Various embodiments of the present invention provide for methods to identify methanogens in biological samples, such as, but not limited to stool. In various embodiments, the biological sample could be a mucosal biopsy or aspirated fluid taken from any part of the gastrointestinal tract. In various embodiments biopsies or aspirate can be taken from the colon, or stomach, or from the duodenum and the small bowel.

In various embodiments, the sample can be analyzed by culture techniques. In further embodiments, a sample can be subjected to amplification techniques, such as, but not limited to polymerase chain reaction (PCR) to identify the presence of nucleic acids indicative of methanogenic archaea. For example, PCR targeted to the functional gene mcrA, encoding methyl coenzyme M reductase, and 16S rRNA genes, have been used to identify the presence of methanogens. (21).

Various embodiments provide for a method of treatment that can improve at least one symptom or indicator of disease in a patient with chronic lung disease by reduction of methanogens and/or methane production in the patients' digestive tract. Examples of such patients include but are not limited to those who have or are suspected to have asthma, COPD, asthma-COPD overlap, cystic fibrosis, pulmonary fibrosis or restrictive lung disease. The evaluation and diagnostic methods used in the assessment of patients who are suspected to have asthma, COPD, asthma-COPD-overlap and other chronic lung disease conditions are known to those skilled in the art. The symptoms that can be improved include shortness of breath, cough and wheezing. Indicators of disease include methane levels in biological samples and lung function tests. In some embodiments, the use of rescue medication, such as those in an asthma inhaler is reduced or eliminated by the treatments described herein.

In various embodiments of the methods, a therapy could be a prebiotic, such as an oligosaccharide including galactooligosaccharides, fructooligosaccharides, oligofructose, chicory fiber and inulin, which encourage growth of healthy bacteria that can reduce/inhibit the growth of methanogens by competing with them for growth. Alternatively, probiotics containing these beneficial bacteria themselves can also be administered to reduce/inhibit the growth of methanogens by competing with them for growth. In some embodiments the probiotic agent could include, but not limited to, Bifidobacterium sp. or Lactobacilus strains, or L. acidophilus, or Saccharomyces species, L. casei. Both prebiotic and probiotic agents can be consumed as food supplements. For example, a number of plants, including but not limited to root vegetables, such as wild and cultivated yams, wheat, onions, garlic bananas, leeks, artichokes, jicama and agave are known to contain high levels of prebiotics. Similarly, foods such as yogurt, other cultured milk products or even uncultured milk, can provide probiotics. Alternatively, both prebiotics and probiotics can be provided as nutritional supplements or provided in a pharmaceutically packaged form. Thus, in some embodiments, an initial therapy could be a dietary additive that can inhibit the growth of methanogens. In a further embodiment, the therapy could include a liquid formula, such as but not limited to, an elemental formula (for example that sold under the trade name (VIVONEX) or polymeric formula (such as those sold under the trade names BOOST or ENSURE). The product sold under the name VIVONEX is absorbed in the proximal gut. Thus, when this product is the only food given, bacteria in the distal small bowel, and in particular the colon, are deprived of fermentable material to serve as nutrients as their source of energy. Semi-elemental formulas that provide peptides of varying chain length, siple sugars, glucose polymers or starch and fats, including medium chain triglycerides, can also be effective. An effective amount of these prebiotic and probiotic formula can be readily determined depending on the source as effective to reduce the level of methane production in the patient's digestive tract.

In various embodiments, antibiotics, single or in combination, can be used to reduce or eradicate the population of methanogens in the gut. Antibiotics that are poorly absorbed systemically when taken orally, such as rifaximin, bicozamycin, nifuroxazide and aminoglycosides, such as neomycin, and therefore work primarily in the lumen of the large and small intestine can be particularly effective. Antibiotics that are “poorly absorbed systemically when taken orally” include any of those that result in concentrations in peak concentrations in the blood of the patient less than twenty times the level achieved by any of rifaximin, aztreonam bicozamycin, nifuroxazide and neomycin. For example, rifaximin at 550 mg dosage has been shown to result in a mean peak plasma concentrate of from 2.4 to 4 ng/ml. Thus, this drug results in 0.0044 to 0.0073 ng/ml in plasma for each mg of antibiotic ingested. Therefore, some examples of other antibiotics that are poorly absorbed systemically will produce peak plasma concentrations of 0.088 to 0.146 ng/ml for each mg of antibiotic ingested.

Metronidazole, metronidazole esters and/or isomers, can also be used. Further examples of antibiotics include but are not limited to aminoglycosides (e.g., amikacin, gentamicin, kanamycin, neomycin, netilmicin, streptomycin, tobramycin, paromomycin), ansamycins (e.g., geldanamycin, herbimycin) , carbacephems (e.g., loracarbef), carbapenems (e.g., ertapenem, doripenem, imipenem , cilastatin, meropenem), cephalosporins (e.g., first generation: cefadroxil , cefazolin, cefalotin or cefalothin, cefalexin; second generation: cefaclor, cefamandole, cefoxitin, cefprozil, cefuroxime; third generation: cefixime, cefdinir, cefditoren, cefoperazone, cefotaxime, cefpodoxime, ceftazidime, ceftibuten, ceftizoxime, ceftriaxone; fourth generation: cefepime; fifth generation: ceftobiprole), glycopeptides (e.g., teicoplanin, vancomycin), macrolides (e.g., azithromycin, clarithromycin, dirithromycin, erythromycin, roxithromycin, troleandomycin, telithromycin, spectinomycin), monobactams (e.g., aztreonam), penicillins (e.g., amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin), antibiotic polypeptides (e.g., bacitracin, colistin, polymyxin b), quinolones (e.g., ciprofloxacin, enoxacin, gatifloxacin, levofloxacin, lomefloxacin, moxifloxacin, norfloxacin, ofloxacin, trovafloxacin), rifamycins (e.g., rifampicin or rifampin, rifabutin, rifapentine, rifaximin), sulfonamides (e.g., mafenide, prontosil, sulfacetamide, sulfamethizole, sulfanilamide, sulfasalazine, sulfisoxazole, trimethoprim, trimethoprim-sulfamethoxazole (co-trimoxazole, “tmp-smx”), and tetracyclines (e.g., demeclocycline, doxycycline, minocycline, oxytetracycline, tetracycline) as well as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin combination, and tinidazole. Such therapy may be provided for various periods, preferably 1 to 90 days, including periods of 1, 2, 3, 4, 5, 6, 7, 8, 9, 10, 14, 28, 30, 60 or 90 days or even longer, and the treatment may be repeated after stopping treatment for a period for any period, such as 1 to 365 days, if methanogens recur in the gut.

In various embodiments, the antibiotic agent is rifaximin, provided in a total daily dose of 200-2400 mg, such as 400 to 1200 mg, taken in divided doses 2-3 times a day. In various embodiments, the medication can be taken from 5 days to 28 days, and be repeated after stopping treatment for any length of time. In various embodiments, rifaximin can be combined with other antibiotics

In various embodiments, the antibiotic agent is neomycin, provided at a dose of 50 mg to 1000 mg, taken up to 4 times a day. In various embodiments, the medication can be taken from 5 days to 28 days, and be repeated after a while. In various embodiments, neomycin can be combined with other antibiotics.

In various embodiments, the antibiotic agent is metronidazole, provided at a dose of 50 mg to 1000 mg, taken up to 4 times a day. In various embodiments, the medication can be taken from 5 days to 28 days, and be repeated after a while. In various embodiments, metronidazole can be combined with other antibiotics.

Dosages for other antibiotic agents that can reduce the level of methanogenic archae in the patient's digestive tract can be determined based on effective doses for other treatments and titrating the patient until methane levels are reduced.

Statin agents (HMG-CoA reductase inhibitors) can also lower methanogeneis by either inhibiting the growth of archaea, including methanogens, or by directly inhibiting methane production. Advantageously, these agents can inhibit archaeal cell membrane biosynthesis without affecting bacterial numbers. Lovastatin lactone is believed to be particularly effective at inhibiting methanogenesis directly. Other statins, including but not limited to mevastatin, pravastatin, atorvastatin, rosuvastatin and simvastatin, in lactone, acid and hydroxyl acid forms can also be effectively used to lower methane production in patients having chronic lung disorders and diseases.

Statin agents can be administered in the same dosages and frequency as prescribed for other purposes, such as control of cholesterol. However, in some embodiments, the dosages can be reduced by up to one-twentieth of the dosage typically prescribed for cholesterol treatment or increased by up to ten times such dosage. Typical dosages for statin drugs are well-known, such as are described in an original investigation published in JAMA by Rodriguez et al. (22), the disclosure of which is incorporated by reference in accordance with its listing in the References section of this specification.

### EXAMPLE

A prospective, open label pilot study will be performed. Based on results presented in Table 1 showing the presence of methane production in over 20% of patients with lung disease, it is expected that 50 treated subjects will be effective to show a significant positive effect on pulmonary function testing (PFT). Assuming a 20% drop rate will require the enrollment of 65 patients who are methane positive.

Data will be collected from symptomatic but stable patients with chronic lung disease and reactive airways aged 18-80 years with a diagnosis of chronic obstructive pulmonary disease based on American Thoracic Society/European Respiratory Society (ATS/ERS) criteria (3). Clinical, physiologic and imaging features are used for conformation of diagnosis. History will include smoking (past, current, never). Patients with post-tuberculosis airflow limitation, infiltrative respiratory diseases, chest wall and neuromuscular disorders, left ventricular heart failure, those unable to perform lung function testing according to ATS guidelines, collagen vascular disease and those with reduced GFR will be excluded. Pregnant and lactating women will be excluded as well, and all women of child-bearing potential will have a urinary pregnancy test performed prior to taking the drugs. Three hundred patients will be screened by breath test, in order to enroll 65 patients in the study, who are found to be methane positive (>10 PPM) by breath test. As a result, this design will yield 2 groups of patients: (A) patients who are methane producers who do show a methane response to antibiotic treatment; (B) patients who are methane producers who do not show a methane response to antibiotic treatment.

PFT will be done at the pulmonary physiology, according to standard criteria. PFT will be performed in seated position, consisting of post-bronchodilator spirometry (which includes slow vital capacity, SVC), lung volume subdivisions measured by body plethysmography, and single-breath diffusion capacity (DLCO) and according to American Thoracic Society guidelines (3). Patients will meet criteria of post-bronchodilator ratio of forced expiratory volume at one second (FEV1) to forced vital capacity (FVC) less than 0.7 to qualify for the ATS definition of chronic obstructive pulmonary disease (COPD) (5). Patients with 12% or greater and 200 mL increase in FEV1 after bronchodilators will be defined as having asthma. Patients with the same response to bronchodilators and imaging findings of emphysema will be classified as the asthma-COPD overlap syndrome (3,5). Predicted values for spirometric indices, lung volumes and single-breath carbon monoxide diffusion capacity will be from Morris (43). Predicted values for inspiratory capacity (IC) will be derived from Bates (44). PFT's performed within 1 year of the prior to encounter will be considered valid. The COPD Assessment Test (CAT) will be used to assess degree of physical impairment (45). Dyspnea will be graded according to 3 grades of the MMRC scale (0, 2 and 4) based on a questionnaire administered by the laboratory technologist (46). Severity of COPD will graded based on the GOLD 2017 staging (3).

Testing will be measured using a standard breath test analysis, as done routinely in the GI motility laboratory however, without lactulose. Patients will be asked to inhale deeply, hold the breath for 15 seconds and then exhale into the bag. A level of exhaled breath methane (eCH4) of ≥10 PPM is considered to be positive (35). Patients who were treated with antibiotics within one month prior to the screening will be excluded. While breath test is normally done during fasting, using a non-absorbable carbohydrate load (such as lactulose), per logistic reasons subjects will be non-fasting, but will be instructed to consume similar food during the repeated test, which preferably should be conducted at the same time of the day. CO2 and nitric oxide will be measured as well.

Methane producers will be treated with a course of rifaximin 550 mg TID PO and metronidazole 250 mg TID PO, both for 10 days. A regimen of antibiotics with neomycin instead of metronidazole was shown to eradicate methane production by breath test in 87% of subjects (40). Replacement of neomycin by metronidazole will have a similar effect (personal communication).

Patients seen in the pulmonary clinic fulfilling study criteria will be asked to participate, and after signing an informed consent form will be asked to provide a breath sample and will complete validated pulmonary questionnaires. All patients will be provided with the appropriate antibiotics. Two to three weeks after completion of the course of antibiotic therapy, subjects will have repeated breath test, PFT and symptom assessment using similar questionnaire

The scores of the questionnaires and the FEV1/FVC metric of the PFT's will serve as primary outcomes. Other metrics of PFTs; FEV1, FVC and FEF25-75 (which is an index of small airway function and recently has been used to assess airway response to meds will be used as secondary outcomes. Each patient will have their MMRC and CAT scores compiled and differences in the scores between cohorts will be measured by nonparametric methods; Mann-Whitney U test and Kruskal-Wallis test and PFT's will be analyzed using Student-t test. Symptoms and variables of PFT will be assessed before and after therapy in each patient. Patients in whom antibiotic therapy fails to eliminate methane production will serve as a control group (46).

Sensitivity analyses will be performed using G*Power to estimate the detectible difference PFT between those anticipated to have methane eradicated versus those who do not have it eradicated. 80% of the sample (N=40) will have eradicated methane, and 20% will not (N=10). The detectible difference is d=1.01 with the proposed sample of N=50, α=0.05, and 80% power. This translates into a large difference between groups.

The results will show that methane positive patients with lung disease will have a significant positive effect on PFT upon receiving the antibiotic therapy.

Although the foregoing description describes certain embodiments in detail, those skilled in the art will appreciate that modifications of the description can be made without departing from the inventive concepts described. Accordingly, the foregoing description should not be viewed as limiting the invention to only those specific details described.

### REFERENCES

The following publications are hereby incorporated by reference in their entirety:

1) National Asthma Education and Prevention Program: Expert panel report III: Guidelines for the diagnosis and management of asthma. Bethesda, Md.: National Heart, Lung, and Blood Institute, 2007. (NIH publication no. 08-4051)
2) National Institute for Health and Care Excellence (NICE). Asthma: diagnosis, monitoring and chronic asthma management.
3) Vogelmeier C F, Criner G J, Martinez F J, et al. Global strategy for the diagnosis, management, and prevention of chronic obstructive lung disease 2017 Report. GOLD Executive Summary. Am J Respir Crit Care Med 2017; 195(5): 557-582.
4) Rennard S, Thomashow B, Crapo J, Yawn B, McIvor A, Cerreta S, Walsh J, Mannino D. Introducing the COPD Foundation Guide for Diagnosis and Management of COPD, recommendations of the COPD Foundation . COPD. 2013 Jun; 10(3): 378-89.
5) Pellegrino R, Viegi G, Brusasco V, et al. Interpretative strategies for lung function tests. Am J Respir Crit Care Med 2005; 26: 948-968.
6) Neish AS. Microbes in gastrointestinal health and disease. Gastroenterology 136: 65-80, 2009.
7) Sekirov I, Russel S L, Caetano L M, Antunes M, Brett Finlay B. Gut Microbiota in Health and Disease. Physiol Rev 90: 859-904,2010
8) Chassard C, Lacroix C. Carbohydrates and the human gut microbiota. Curr Opin Clin Nutr Metab Care 2013; 16: 453-460.
9) Sahakian A B, Jee S R, Pimentel M. Methane and the gastrointestinal tract. Dig Dis Sci 2010; 55: 2134-2143
10) Pochart P, Lémann F, Flourié B, Pellier P, Goderel I, Rambaud J C. Pyxigraphic sampling to enumerate methanogens and anaerobes in the right colon of healthy humans. Gastroenterology 1993; 105: 1281-1285.
11) Dridi B, Henry M, El Khéchine A, Raoult D, Drancourt M. High prevelance of Methanobrevibacter smithii and Methanosphaera stadtmanae detected in the human gut using an improved DNA detection protocol. PLoS ONE 2009; 4: e7063.
12) Levitt M D, Fume J K, Kuskowski M, Ruddy J. Stability of human methanogenic flora over 35 years and a review of insights obtained from breath methane measurements. Clin Gastroenterol Hepatol 2006; 4: 123-129.
13) Kim E J, Paik C N, Chung W C, Lee K M, Yang J M, Choi M G. The characteristics of the positivity to the lactulose breath test in patients with abdominal bloating. Eur J Gastroenterol Hepatol 2011; 23: 1144-1149
14) Pimentel M, Lin H C, Enayati P, et al. Methane, a gas produced by enteric bacteria, slows intestinal transit and augments small intestinal contractile activity. Am J Physiol Gastrointest Liver Physiol. 2006; 290; G1089-G1095.
15) Jahng J, Jung I S, Choi E J, Concklin J L, Park H. The effects of methane and hydrogen gases produced by enteric bacteria on ileal motility and colonic transit time. Neurogastroenterol Motil. 2012; 24; 185-190.
16) Park Y M, Lee Y J, Hussain Z, Lee Y H, Park H. The effects and mechanism of action of methane on ileal motor function. Neurogastroenterol Motil. 2017; 29: e13077.
17) Triantafyllou K, Chang K and Pimentel M. Methanogens, Methane and Gastrointestinal Motility. J Neurogastroenterol Motil 2014; 20(1): 31-40.
18) Triantafyllou K, Chang K and Pimentel M. Methanogens, Methane and Gastrointestinal Motility. J Neurogastroenterol Motil 2014; 20(1): 31-40.
19) Ghosal U C, Srivastava D, Misra A. A randomized double-blind placebo-controlled trial showing rifaximin to improve constipation by reducing methane production and shortening colon transit time: a pilot study. Indian J Gastroenterol. 2018; 37: 416-423.
20) Gottlieb K., Wacher V., Sliman J., Pimentel M. Review article: inhibition of methanogenic archaea by statins as a targeted management strategy for constipation and related disorders. Alimentary Pharmacology & Therapeutics, 2015; 43: 197-212.

### 21) Vianna, M. E., Conrads, G., Gomes B. P. F. A., Horz, H. P. Identification and Quantification of Archaea Involved in Primary Endodontic Infections, 2006; 44: 1274-1282.

22) Rodrigues F., Maron, D., Knowles, J. Association between intensity of statin therapy and mortality in patients with atherosclerotic cardiovascular disease, JAMA Cardiology, 2017; 2: 47-54.
23) Forterre P, Brochier C, Philippe H. Evolution of the archaea. Theor Popul Biol 2002; 61: 409-422.
24) Khelaifia S, Drancourt M. Susceptibility of archaea to antimicrobial agents: applications to clinical microbiology. Clin Microbiol Infect 2012; 18: 841-848
25) Eckburg P B, BiK E M, Bernstein C N, et al. Diversity of the human intestinal microbial flora. Science 2005; 308: 1635-1638.
26) Lepp P W, Bring M M, Ouverney C C, Palm K, Armitage G C, Relman D A. Methanogenic archaea and human periodontal disease. Proc Natl Acad Sci USA 2004; 101: 6176-6181.
27) Miller T L, Wolin M J. Enumeration of Methanobrevibacter smithii in human feces. Arch Microbiol 1982; 131: 14-18.
28) Matarazzo F, Ribeiro AC, Faveri M, Taddei C, Martinez M B, Mayer M P. The domain Archaea in human mucosal surfaces. Clin Microbiol Infect 2012; 18: 834-840.
29) Bond J H Jr, Engel R R, Levitt M D. Factors influencing pulmonary methane excretion in man. J Exp Med 1971; 133: 572-588.
30) Florin T H, Zhu G, Kirk K M, Martin N G. Shared and unique environmental factors determine the ecology of methanogens in humans and rats. Am J Gastroenterol 2000; 95: 2872-2879.
31) Christl S U, Murgatroyd P R, Gibson G R, Cummings J H. Production, metabolism and excretion of hydrogen in the large intestine. Gastroenterology 2000; 102(4 Pt 1): 1269-1277.
32) Pimentel M, Mayer A G, Park S, Chow E J, Hasan A, Kong Y. Methane production during lactulose breath test is associated with gastrointestinal disease presentation. Dig Dis Sci 2003; 48: 86-92.
33) Hwang L, Low K, Khoshini R, et al. Evaluating breath methane as a diagnostic test for constipation-predominant IBS. Dig Dis Sci 2010; 55: 398-403.
34) McKay L F, Eastwood M A, Brydon W G. Methane excretion in man-a study of breath, flatus and faeces. Gut 1985; 26: 69-74.

### 35) Rezaie A, Buresi M, Lembo A, Lin H , McCallum M, Rao S, Schmulson M, Valdovinos M, Zakko M, Pimentel M. Hydrogen and Methane-Based Breath Testing in Gastrointestinal Disorders: The North American Consensus. . Am J Gastroenterol 2017; 112: 775-784

36) Weaver G A, Krause J A, Miller T L, Wolin M J. Incidence of methanogenic bacteria in a sigmoidoscopy population: an association of methanogenic bacteria and diverticulitis. Gut 1986; 27: 698-704.
37) Blaut M. Metabolism of methanogens. Antoine Van Leeuwenhoek 1994; 66: 187-208.
38) Attaluri A, Jackson M, Valestin J, Rao S S C. Methanogenic flora is associated with altered colonic transit but not stool characteristics in constipation without IBS. Am J Gastroenterol. 2010; 105; 1407-1411.
39) Lee K M, Paik C N, Chung W C, Yang J M, Choi M G. Breath methane positivity is more common and higher in patients with objectively proven delayed transit constipation. Eur J Gastroenterol Hepatol. 2013; 25; 726-732.
40) Chatterjee S, Park S, Low K, Kong Y, Pimentel M. The degree of breath methane production in IBS correlates with the severity of constipation. Am J Gastroenterol. 2007; 102; 837-841.
41) Furnari M, Savarino E, Buzzone L, et al. Reassessment of the role of methane production between irritable bowel syndrome and functional constipation. J Gastrointestin liver Dis. 2012; 21; 157-163.
42) Low K, Hwang L, Hua J, Zhu A, Morales W, Pimentel M. A combination of rifaximin and neomycin is most effective in treating irritable bowel syndrome with methane on lactulose breath test. J Clin Gastroenterol. 2010; 44; 547-550.
43) Morris J. F., Koski A., Johnson L. C. Spirometric standards for healthy nonsmoking adults. Am. Rev. Respir. Dis.10319715767

### 44) Bates D V. Respiratory Function in Disease, Saunders, 1989, pp. 108-109.

45) Lee S-D et al. The COPD assessment test (CAT) assists prediction of COPD exacerbations in high-risk patients. Respir Med 2014; 108 (4): 600-608
46) Mahler D A1, Wells C K. Evaluation of clinical methods for rating dyspnea. Chest 1988 93(3): 580-6.
47) Dixon W J, Massey F J Jr. Introduction to statistical analysis, 4th ed. New York: McGraw-Hill, 1983.

What is claimed is:

1. A method of improving a symptom or indicator of disease in a patient with chronic lung disease, comprising:
identifying the patient as likely to respond to treatment by reduction of methane production and/or methanogenic archaea in the patient's digestive tract by evaluating the patient as methane positive; and
administering one or more agents to the identified methane positive patient, wherein the one or more agents reduce or eliminate the methane production and/or the methanogenic archaea in the patient's digestive tract.

2. The method of claim 1, wherein the method improves an indicator of a disease comprising reduction or elimination of methane gas in the patient's breath.

3. The method of claim 1, wherein the patient is evaluated as methane positive by identifying 1 ppm or greater of methane in the patient's breath.

4. The method of claim 1, wherein the patient is evaluated as methane positive by identifying 10 ppm or greater of methane in the patient's breath.

5. The method of claim 1, wherein the method improves an indicator of a disease comprising a pulmonary function test.

6. The method of claim 1, wherein the agent comprises a probiotic or a prebiotic.

7. The method of claim 1, wherein the agent is in an elemental, semi-elemental or polymeric formula.

8. The method of claim 6, wherein the agent is present in a food.

9. The method of claim 1, wherein the agent comprises an antibiotic that is poorly absorbed systemically.

10. The method of claim 9, wherein the agent is selected from the group consisting of rifaximin and an aminoglycoside.

11. The method of claim 1, wherein the agent is administered daily.

12. The method of claim 11, wherein the agent is administered for at least 5 days.

13. The method of claim 1, further comprising repeating administering the one or more agents after stopping treatment for at least one day or longer.

14. The method of claim 1, wherein the symptom is selected from the group consisting of shortness of breath, cough and wheezing.

15. The method of claim 1, wherein the method reduces methane levels in the subject.

16. The method of claim 1, wherein the method reduces methanogenic archaea in the subject's intestine.

17. The method of claim 1, wherein the method reduces methane levels as assessed by breath testing.

18. The method of claim 1, wherein the method further comprises evaluating intestinal methanogenic archaea or breath methane, before, after or during treatment.

19. The method of claim 1, wherein the method reduces or eliminates the use of rescue medication by the subject.
